(19) United States
(12) Patent Application Publication
(10) Pub. No.: US 2024/0050716 A1
(43) Pub. Date: Feb. 15, 2024

(54) BALLOON CATHETER WITH BIASING MEMBER

(71) Applicant: Jihad Ali Mustapha, Ada, MI (US)

(72) Inventor: Jihad Ali Mustapha, Ada, MI (US)

(21) Appl. No.: 18/448,736

(22) Filed: Aug. 11, 2023

Related U.S. Application Data
(60) Provisional application No. 63/371,289, filed on Aug. 12, 2022.

(51) Int. Cl. A61M 25/10

(52) U.S. Cl. CPC A61M 25/104; A61M 25/10181; A61M 2025/1068

(57) ABSTRACT

A balloon catheter includes an elongate shaft, a balloon, and one or more biasing members. The elongate shaft has a proximal end and a distal end. The balloon is attached to the elongate shaft at the distal end of the elongate shaft. The balloon includes a balloon chamber for receiving inflation fluid. The one or more biasing members are connected to the balloon. The one or more biasing members move to a non-biased state when the balloon is inflated and move to a biased state when the balloon is deflated such that the one or more biasing members cause the balloon to return to an original shape of the balloon.

### CROSS-REFERENCE TO RELATED APPLICATIONS

This application claims priority to U.S. Provisional Application No. 63/371,289 filed Aug. 12, 2022, the entire contents of which is hereby incorporated by reference.

### TECHNICAL FIELD

The present invention relates to balloon catheters, and, more particularly, to a balloon catheter having a biasing member.

### BACKGROUND

Balloon angioplasty is a procedure used to open narrowed or blocked arteries in many parts of the body. For example, balloon angioplasty can be performed in tibial and peroneal arteries, the femoral artery, the popliteal artery, the iliac artery, the aorta, coronary arteries, carotid arteries, or any other artery in the body. During an angioplasty procedure, a catheter that has a small balloon on its tip around a shaft is inserted into the narrowed or blocked artery. The balloon is inflated with an inflation fluid at the blockage site in the artery to flatten or compress the plaque against the artery wall. The balloon is then deflated. During a balloon angioplasty, the balloon may be inflated and deflated several times. At the conclusion of the procedure, the balloon is then deflated and removed from the artery.

### BRIEF SUMMARY OF THE INVENTION

According to one embodiment, a balloon catheter comprises an elongate shaft having a proximal end and a distal end, a balloon attached to the elongate shaft at the distal end of the elongate shaft, the balloon including a balloon chamber for receiving inflation fluid, and one or more biasing members connected to the balloon, the one or more biasing members move to a non-biased state when the balloon is inflated and move to a biased state when the balloon is deflated such that the one or more biasing members cause the balloon to return to an original shape of the balloon.

According to another embodiment, a balloon catheter comprises an elongate shaft having a proximal end and a distal end, a balloon attached to the elongate shaft at the distal end of the elongate shaft, the balloon including a balloon chamber for receiving inflation fluid, and one or more biasing members connected to the balloon and wrapped around the elongate shaft, the one or more biasing members include one or more helical sections having one or more cells, wherein the one or more biasing members move to a non-biased state when the balloon is inflated and move to a biased state when the balloon is deflated such that the one or more biasing members cause the balloon to return to an original shape of the balloon.

According to yet another embodiment, a balloon catheter comprising an elongate shaft having a proximal end and a distal end, a balloon attached to the elongate shaft at the distal end of the elongate shaft, the balloon including a balloon chamber for receiving inflation fluid, and the balloon including an inner balloon layer and an outer balloon layer, and one or more biasing members connected to the balloon and wrapped around the inner balloon layer, the one or more biasing members include one or more helical sections having one or more cells, wherein the one or more biasing members expand when the balloon is inflated and compress when the balloon is deflated such that the one or more biasing members force the inflation fluid out of the balloon chamber.

Additional features, advantages, and embodiments of the invention are set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1A illustrates an exemplary balloon catheter having a balloon, according to an embodiment of the invention.

FIG. 1B is a schematic view of the balloon of FIG. 1A in a deflated state, according to an aspect of the disclosure.

FIG. 1C is a schematic side view of another balloon in a deflated state, according to another embodiment.

FIG. 1D is a schematic view of the balloon of FIG. 1C in a vessel during a balloon angioplasty, according to an aspect of the disclosure.

FIG. 2A is a schematic view of a balloon having a biasing member, according to an aspect of the disclosure.

FIG. 2B is a schematic view of the balloon of FIG. 2A in a deflated state, according to an aspect of the disclosure.

FIG. 3 is a schematic view of another balloon having a biasing member, according to another embodiment.

FIG. 4 is a schematic view of another balloon having a biasing member, according to another embodiment.

FIG. 5 is a schematic view of a balloon having a biasing member and being in a deflated state, according to another embodiment.

FIG. 6 is a schematic view of a spring isolated from the balloon in FIG. 5, according to an aspect of the disclosure.

FIG. 7 is a schematic view of the balloon of FIG. 5 in an inflated state, according to an aspect of the disclosure.

FIG. 8 is a schematic view of another balloon having a biasing member and being in a deflated state, according to another embodiment.

FIG. 9 is a schematic view of the balloon of FIG. 8 in an inflated state, according to aspects of the disclosure.

FIG. 10 is a schematic view of another balloon having a biasing member and being in a deflated state, according to another embodiment.

FIG. 11 is a schematic view of the balloon of FIG. 10 in an inflated state, according to aspects of the disclosure.

FIG. 12 illustrates an exemplary balloon catheter having a balloon including a biasing member and being in an inflated state, according to another embodiment.

FIG. 13 illustrates the balloon catheter having a balloon of FIG. 12 in a deflated state, according to an aspect of the invention.

FIG. 14 illustrates an exemplary balloon catheter having a balloon including a biasing member and being in an inflated state, according to another embodiment.

FIG. 15 illustrates an exemplary balloon catheter having a balloon including a biasing member and being in an inflated state, according to another embodiment.

### DETAILED DESCRIPTION OF THE INVENTION

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though they may. Furthermore, the phrases “in another embodiment” and “in some other embodiments” as used herein do not necessarily refer to a different embodiment, although they may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, terms such as “comprising,” “including,” and “having” do not limit the scope of a specific claim to the materials or steps recited by the claim.

Approximating language, as used herein throughout the specification and claims, is applied to modify any quantitative representation that could permissibly vary without resulting in a change in the basic function to which it is related. Accordingly, a value modified by a term or terms, such as “about,” “approximately,” “generally,” and “substantially” is not to be limited to the precise value specified. In at least some instances, the approximating language may correspond to the precision of an instrument for measuring the value, or the precision of the methods or the machines for constructing the components and/or the systems or manufacturing the components and/or the systems. For example, the approximating language may refer to being within a one, a two, a four, a ten, a fifteen, or a twenty percent margin in either individual values, range(s) of values and/or endpoints defining range(s) of values.

There is a need for balloon angioplasty throughout various parts of the anatomy and vasculature, such as at appendages, cardiovascular areas, neurovascular areas, etc. For example, balloon angioplasty can be performed in tibial and peroneal arteries, the femoral artery, the popliteal artery, the iliac artery, the aorta, coronary arteries, carotid arteries, or any other artery or vessel in the body. Balloon angioplasty can require precision movements in the tortuous vasculature. Balloons can be inflated and deflated throughout a procedure. In one aspect, for example, there is a need for tibial arterial balloon angioplasty, as well as for pedal arterial and pedal loop balloon angioplasty. The pedal loop is a combination of multiple arteries connected together via a junction located mostly at the level of the distal metatarsal lateral bones in between the first toe and the second toe. The arteries of the pedal loop include the lateral plantar artery from the posterior circulation that comes from the posterior tibial artery. The pedal loop then runs diagonally from the medial plantar artery to the lateral plantar artery and connects to the arcuate artery or the transmetatarsal artery. The arcuate artery or the transmetatarsal artery transverse the foot from the plantar area to the dorsal area and connects with the dorsalis pedis artery. The dorsalis pedis artery is located on top of the foot and is part of the anterior circulation. The dorsalis pedis artery is a continuation of the anterior tibial artery as part of the anterior circulation. Thus, the pedal loop includes the lateral plantar artery, the medial plantar artery, the arcuate artery or transmetatarsal artery, and the dorsalis pedis artery. Accordingly, an intact pedal loop supplies oxygenated blood to the anterior circulation and the posterior circulation and for angiosome distribution. The angiosome is an area defined by the arterial blood supply. Areas on the dorsal aspect of the foot are referred to as anterior distribution angiosomes and areas receiving blood on the bottom of the foot are referred to as posterior angiosome distribution. The tibial arteries and the pedal arteries detailed above can be treated with balloon angioplasty. Digital arteries can also be treated with balloon angioplasty. Digital arteries supply blood to the toes. Digital arteries include anterior digital arteries and posterior digital arteries that receive blood from the anterior circulation and the posterior circulation, respectively.

Tibial-pedal disease can be long and complex and may include lesions (e.g., blockage) having lengths in the range of three hundred millimeters to four hundred millimeters. Longer lesions are present when the blockage involves pedal loop blockage. The average length of the pedal loop is in the range of two hundred millimeters to three hundred millimeters based on the height and the sex of the patient. Balloons used in balloon angioplasty for the pedal loop can include a treatment length up to three hundred millimeters. Inflation and deflation may be required throughout the procedures. Current balloons have slow inflation and deflation times. Shorter balloons tend to deflate faster than longer balloons. Further, balloons with larger diameters take longer to inflate and to deflate. This increased inflation and deflation time of the longer balloons, as compared to shorter balloons, increases the amount of time that the catheter is in the vein, thereby increasing an amount of radiation exposure of the patient, and increasing an amount of time that the artery or the vein is occluded. Similarly, balloons with larger diameters require more time to achieve the nominal pressure which determines the actual diameter of the balloon. The longer it takes it takes to inflate a balloon, the longer it takes to deflate the balloon. Currently there are no methods to enhance balloon inflation or deflation (e.g., to reduce the amount of time to inflate or to deflate the balloon). Additionally, current balloons do not re-wrap when the balloon is deflated and form a “wing” that could irritate the just treated artery due to long helical wings.

The embodiments of the present disclosure provide for a spring system for a balloon that is used to decrease the deflation time and return the balloon to substantially the original shape of the balloon prior to utilization. The embodiments of the present disclosure may be used for any balloon catheter. For example, the embodiments detailed herein can be used in the treatment of carotid artery disease. Carotid arteries are the two main arteries that supply blood flow to the brain. Any interruptions of blood flow in the carotid arteries that are longer than two to three minutes can cause damage to the brain. For example, interruptions of the blood flow in the carotid arteries leads to brain cell deprivation of blood and oxygen which leads to death of the brain cells unless the obstructive nature of the carotid artery is removed and the blood to the brain is restored.

The decreased deflation time provided by the embodiments of the present disclosure have a substantial value in reducing the time of obstructive blood flow to the brain. In situations where a dissection or a perforation is present at the site of treatment of the carotid arteries, the embodiments of the balloon of the present disclosure provides an excellent and quick inflation to help correct the arterial wall dissection and helps to stop blood extravasation at the same time. While the balloon is preventing further deterioration of the complication, a solution is usually being prepared to bring up to the complication site when blood flow is urgently needed distal to the site where the balloon is inflated. The fast deflation of the balloon of the present disclosure is particularly beneficial in areas such as the carotid arteries (e.g., the arteries that supply blood to the brain). The fast deflation provided by the embodiments of the present disclosure enhances the quick re-establishment of blood flow to the brain.

The embodiments of the present disclosure also eliminate the “wings” formed by incomplete deflation of the current balloons that could cause dislodgements of loose plaques while pulling the balloon out from the area treated. The embodiments of the balloon of the present disclosure reduce the presence of wing formation to substantially zero or non-existent and provide for substantially complete deflation of the balloon due to the combined spring system. The spring system retains kinetic energy due to the mechanical inflation of the balloon which causes the spring system to stretch or compress. While the balloon is inflated, the energy in the spring system is stored and is ready to release the stored energy by means of the spring systems moving back to the baseline shape of the spring system. For example, when the spring system includes a baseline shape that is elongated or stretched, the spring system is compressed when the balloon is inflated. When the spring system includes a baseline shape that is compressed, the spring system is elongated or stretched when the balloon is inflated. The atmospheric pressure within the balloon keeps the spring compressed (or elongated) as long as needed and the energy stored in the spring system is generated by the mechanical inflation of the balloon and released by the mechanical deflation of the balloon. This closed and self-enclosed circle of energy generation, storage, and release of the spring system provides for an improved mechanism for returning the balloon to substantially an original shape of the balloon. The generated and stored energy is used to help resolve a long-term problem of balloons that form “wings” when deflated and provides additional options for relatively longer balloons as well as with tapered balloons with significant variations in the diameter of the balloon, as detailed herein.

The embodiments of the present disclosure may also be used in aortic valve balloons. Aortic valve balloons are a critical part of a critical procedure where inflation and deflation make a significant impact to a patient's hemodynamics. During the inflation time of an aortic valve balloon, the left ventricle outflow to the rest of the body is obstructed. The aortic valve balloon must be deflated as fast as possible to restore blood flow from the left ventricle to the rest of the body. Current aortic valve balloons have a larger diameter balloon disposed around a smaller diameter balloon. During the deflation of such balloons, the larger diameter balloon deflates quickly in an initial deflation phase and the deflation of the larger diameter balloon cascades until the larger diameter balloon contacts the smaller diameter balloon. This initial deflation is followed with a slow deflation of the rest of the deflation phase. In addition to the delayed deflation time, current aortic valve balloons tend to form wrinkles and wings as the balloon recoils. In this way, the balloon does not return to an original (e.g., base line) diameter of the balloon and the wrinkles or wings cause the balloon to bunch up. The wrinkles and wings occupy a part of the aortic valve orifice which may cause obstruction to the blood stream from the left ventricle to the rest of the body. Thus, providing a balloon that deflates faster than the current balloons is especially important in aortic valve applications. Therefore, the embodiments of the present disclosure provide for a balloon that does not develop wrinkles and wings and thus reduces the obstruction time of the aortic valve orifice when the balloon is deflated.

When the spring is in an expanded state, the spring harnesses mechanical energy that is maintained by the mechanical inflation of the balloon atmospheric pressure causing inequality in forces. Thus, the mechanical balloon inflation generates a greater force compared to the force generated by the expansion of the spring. The greater force of the mechanical balloon inflation keeps the spring expanded as the force of the spring is less than the force of the inflated balloon. As long as the mechanical balloon inflation is maintained, the energy (e.g., mechanical energy or force) stored in the spring stays in check due to the lower force it contains compared to the inflated balloon. Therefore, the balloon mechanical force during inflation is greater than the force of the spring when the spring is expanded (e.g., the inflated balloon force>spring force) primarily due to the atmospheric pressure generated by the inflation/deflation device. Upon deflation of the balloon via removing the atmospheric pressure by a mechanical negative pressure generated by the inflation/deflation device, an immediate reverse in force equality happens so that the spring force is greater than the force of the deflated balloon (e.g., spring force>deflated balloon force) such that the spring retracts or otherwise compresses back to its pre-expanded form and causes the balloon to return to its original form faster than a balloon without these features due to the additional mechanical energy stored in the spring. Thus, the deflation time of the balloon is decreased compared to current balloons, and the spring helps to reduce the wing effect of the deflated balloon by pulling the balloon back to its original pre-inflated shape and location on the shaft. As detailed above, the wing effect is a phenomenon that occurs after a balloon is inflated then deflated causing the normally symmetrical circumferential balloon to have waves or wrinkles. A size of the wings of the balloon is greater than the pre-inflation diameter of the symmetrical circumferential balloon shape. Further, the longer the balloon, the more prone the balloon is to forming wings which leads to difficulties in retracting the balloon from the treated target segment into the access sheath.

As detailed above, the balloon in an angioplasty procedure is inflated and deflated. In some instances, the balloon may deflate slowly and/or the balloon may not deflate entirely to its original form. In such instances, the balloon may not entirely separate from the plaque area, the balloon may be difficult to extract from the guide catheter, and/or the balloon may be difficult to re-advance through tight lesions. When deflated, the balloon can flatten and form “wings,” in which the flat, lateral portions of the deflated balloon project laterally outward beyond the catheter. Such flat wings may damage the artery wall as the deflated balloon is advanced through the arterial system into the desired position for inflation. Further, balloon angioplasty for certain arteries, such as the aorta and the carotid artery, requires a relatively fast deflation time of the balloon so as to prevent dangerously blocking the artery with the balloon longer than necessary. The wings may also cause the balloon to wrap around the shaft forming a helical shape. For example, the balloon may twist around the shaft causing the shaft to weaken which leads to a reduced stiffness of the shaft.

Therefore, embodiments of the present disclosure provide for a multiaxial shaft for catheter balloons. For example, the multiaxial shaft includes a spring system. The shaft is able to harness, and store generated mechanical energy during inflation of the balloon (e.g., via the spring system). The stored mechanical energy is then used to aid in deflation of the balloon. For example, when the stored mechanical energy is released, the shaft provides for the following (i) decreased balloon deflation time, (ii) enhanced balloon wrapping close to a baseline of the balloon, (iii) helping to reduce contact between the deflated balloon and the recently treated target segment, especially in instances when the balloon is used for drug delivery to the target vessel wall, (iv) enhanced balloon extraction from access sheaths and guide catheters, and (v) enhanced re-advancement of the balloon into and through tight lesions.

The embodiments of the present disclosure help to reduce wrinkles (e.g., wings) in the balloon that can become an obstacle when the balloon is readvanced through a sheath or a new stenosis (e.g., blockage). For example, the embodiments of the present disclosure provide for reshaping the balloon close to its original shape prior to the initial inflation of the balloon. In this way, the embodiments of the present disclosure can increase the lifespan and the reusability of the balloon, thereby allowing the balloon to be used for multiple procedures and increasing the percentage of crossing sheath valves and new stenosis (e.g., blockages) for which a single balloon can be used.

The spring system is embedded between layers of the balloon catheter and is connected to the fluid dynamic and force generation of the balloon. As the atmospheric pressure increases within the balloon (e.g., from inflation fluid supplied from an indeflator), the pressure causes tension in the spring system, and the spring system moves in a longitudinal direction and/or a circumferential direction and generates and stores negative force or mechanical energy. If the positive atmospheric pressure in the balloon is maintained higher than the generated and stored negative force in the spring system, the balloon inflates. When the positive atmospheric pressure in the balloon is removed via deflation of the balloon, the stored negative force in the spring system is released (almost immediately). This release of the stored negative force helps to deflate the balloon and biases the balloon to its original pre-inflation shape. The relief of the positive atmospheric pressure in the balloon (e.g., when the inflation fluid is removed from the balloon), in combination with the release of the stored negative force of the spring system provides a stable deflation of the balloon and helps to ensure the balloon is deflated to its original shape faster as compared to balloon catheters without the benefit of the present disclosure.

According to one embodiment, the stored negative force is accomplished by combining two layers of balloons over the spring system or other force retention materials (e.g., elastic, or the like). Thus, according to this embodiment, the stored negative force is generated between the walls of the balloon. The balloon catheter also includes larger fluid holes or apertures and a greater number of fluid holes than current balloons, thereby allowing a faster influx of fluid to reduce balloon inflation time as well as reducing balloon deflation time (e.g., reduce the amount of time for inflation and deflation). At the same time, the spring system exerts an inward force from the outer balloon layer toward the inner central portion of the balloon. The added external force from the spring system increases the fluid motion toward the nearest fluid holes. In this way, the fluid holes and the spring system together help to push the fluid into the balloon and remove the fluid from the balloon faster than current balloons regardless of the length or the diameter of the balloon.

The fluid holes, also referred to as apertures, can have a hole diameter in a range of ten thousandths of an inch to forty-five thousandths of an inch (0.0010 inches to 0.0045 inches). The size of the fluid holes is based on the length and the diameter of the balloon. For example, longer balloons or larger diameter balloons can accommodate fluid holes with larger hole diameters (e.g., hole diameters closer to 0.0045 inches) as compared to shorter balloons or smaller diameter balloons that accommodate fluid holes with smaller hole diameters (e.g., hole diameters closer to 0.0010 inches). Adding more fluid holes or increasing the diameter of the fluid holes in current balloons would weaken the structural integrity of the balloon shaft, thereby increasing the likelihood that the wings form in the balloon when the balloon is deflated. Accordingly, the spring system of the present disclosure enables the inflation lumen (e.g., the balloon shaft) to have more fluid holes or larger diameter fluid holes by keeping the structural integrity of the balloon while the balloon is inflated and deflated, as compared to balloon catheters without the benefit of the present disclosure.

The fluid holes in the balloon catheter can be generally circular holes, generally oval holes, or can be generally opened slits that are formed in the inflation lumen of the balloon catheter for directing the fluid from the inflation lumen into the balloon to inflate the balloon. The generally oval holes (e.g., holes with a major axis and a minor axis, the major axis being greater in length than the minor axis) can be used with balloons having a balloon length in a range of twelve centimeters to twenty-four centimeters (12 cm to 24 cm), but can have a balloon diameter that is substantially equal to a balloon diameter of a balloon having generally circular holes. The generally oval shape of the holes helps to increase the influx and efflux of the fluid into and out of the balloon as compared to other hole shapes. The generally opened slits (e.g., openings that are formed by a long, narrow cut in the inflation lumen) can be used for balloons having a balloon diameter in a range of one millimeter to four millimeters (1 mm to 4 mm). The generally opened slits helps to increase the influx and efflux of the fluid into and out of the balloon for smaller diameter balloons (e.g., balloons having a balloon diameter in a range of 1 mm to 4 mm). The generally oval shaped holes and the generally opened slits allow the balloon to be longer as compared to balloons with generally circular holes.

Accordingly, the generally oval holes and the generally opened slits allow the balloon to be inflated and deflated faster than generally circular holes, while the spring system maintains the structural integrity of the balloon, as detailed above. The spring system also adds to the structural integrity of the balloon shaft by keeping the balloon shaft from kinking and helping longer balloons return back to their near normal pre-inflation shape. The combination of the spring system and the increased number of holes or the larger diameter holes within the fluid delivery shaft (e.g., the inflation lumen) enable delivering a greater amount of fluid in a shorter amount of time as compared to balloons without the benefit of the present disclosure. The spring system and the greater number of holes or the larger diameter holes also enables a shorter deflation time as the spring system releases the mechanical energy and a greater amount of the fluid flows out of the balloon through the holes. The combination of the spring system and the larger holes or greater number of holes work together in a symbiotic synchronized manner to reduce the deflation time and allow more fluid to flow into and out of the balloon as compared to balloon without the benefit of the present disclosure.

In some examples, the spring system can be embedded in the pre-formed tubing of the balloon catheter prior to becoming a balloon. In some examples, the spring system can be manufactured with the balloon catheter at an injection molding phase. Regardless of the method of preparing the balloon catheter having the spring system, the end result is to generate and retain force (i.e., a positive force) for the entire time of balloon inflation. The natural recoil of the spring system is referred to as “negative force.” The stretching and expanding of the spring system is referred to as a “positive pressure.”

When the balloon is no longer needed to be inflated, the positive pressure is removed to deflate the balloon. Removing the positive pressure (e.g., removing the inflation fluid from the balloon) causes the spring system to naturally and immediately recoil (e.g., compress) as a negative force. The negative force aids and supports the deflation of the balloon, leading to quicker balloon deflation time and acceleration of the inflation fluid exiting from the balloon toward the negative pressure source (e.g., the indeflator). The inflation fluid exiting from the balloon is further accelerated by the negative force of the spring system until the balloon is completely empty. The combination of negative pressure (e.g., from the indeflator) and negative force (e.g., from the spring system) augments the process of balloon re-prepping and reduces or prevents any “wings” from forming.

The present disclosure includes different embodiments of the spring system for various types of balloon catheters. However, embodiments for a combination of a balloon catheter with a spring system are endless. The different embodiments (including, e.g., different combinations and/or locations of the spring systems), detailed further below with reference to the drawings, can be used for different types of balloons, based on (i) diameter, (ii) length, (iii) type of compliance, (iv) stent deployment balloon, (v) venous balloons, (vi) large caliber venous and arterial balloons, (vii) intracranial balloons, and/or (viii) critical balloon placement, such as structural heart balloons, left main balloons, carotid balloons, or the like.

The mechanical energy (e.g., positive pressure and/or negative pressure) is generated by an indeflator. The indeflator generates positive atmospheric pressure for delivering the inflation fluid into the balloon to inflate the balloon. The positive atmospheric pressure also generates the stored negative force in the shaft of the balloon via the spring system by expanding (or by compressing) the spring system. The embodiments of the present disclosure can be used for any type of balloon catheter used in the human body, as well as any type of balloon catheter used in veterinarian applications.

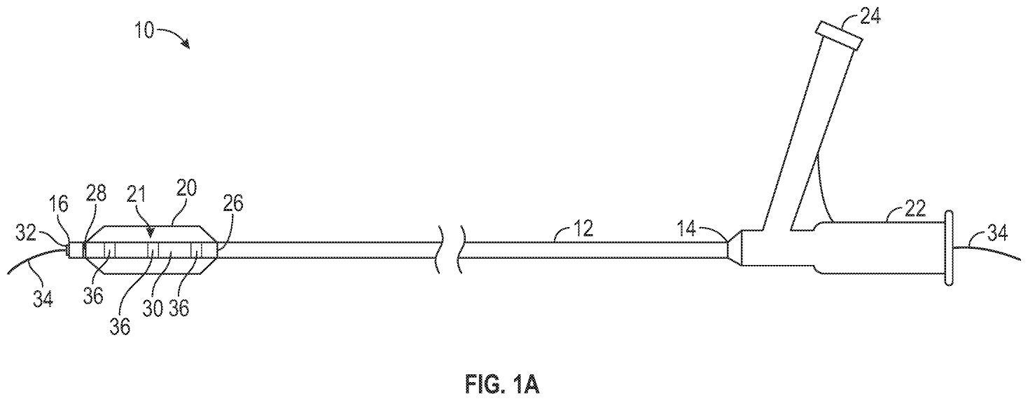

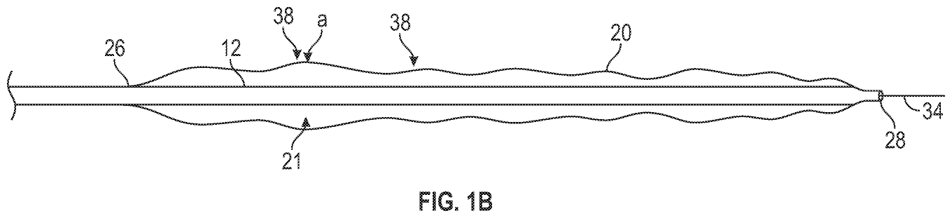

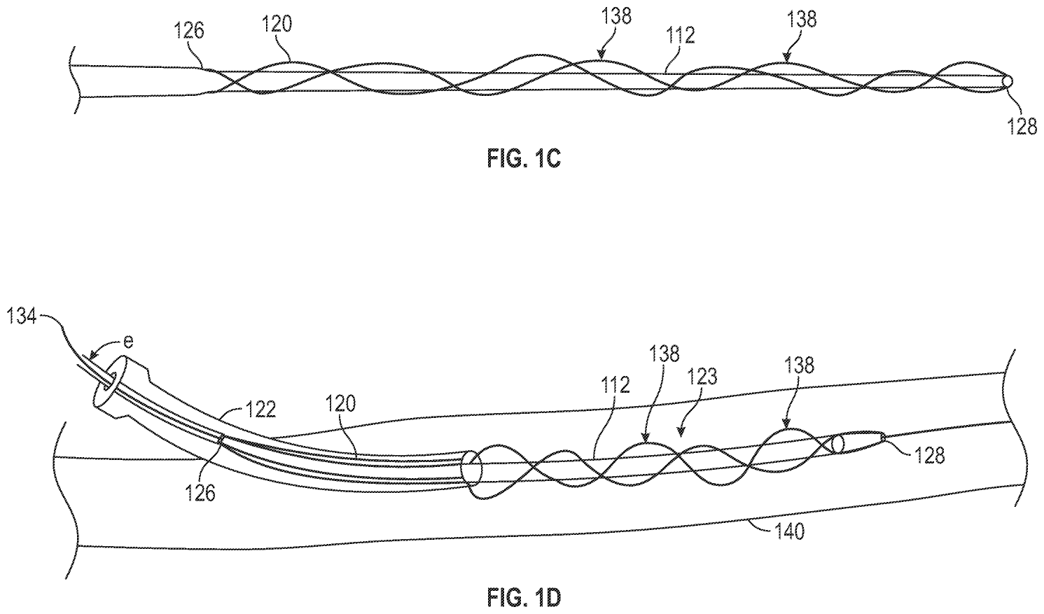

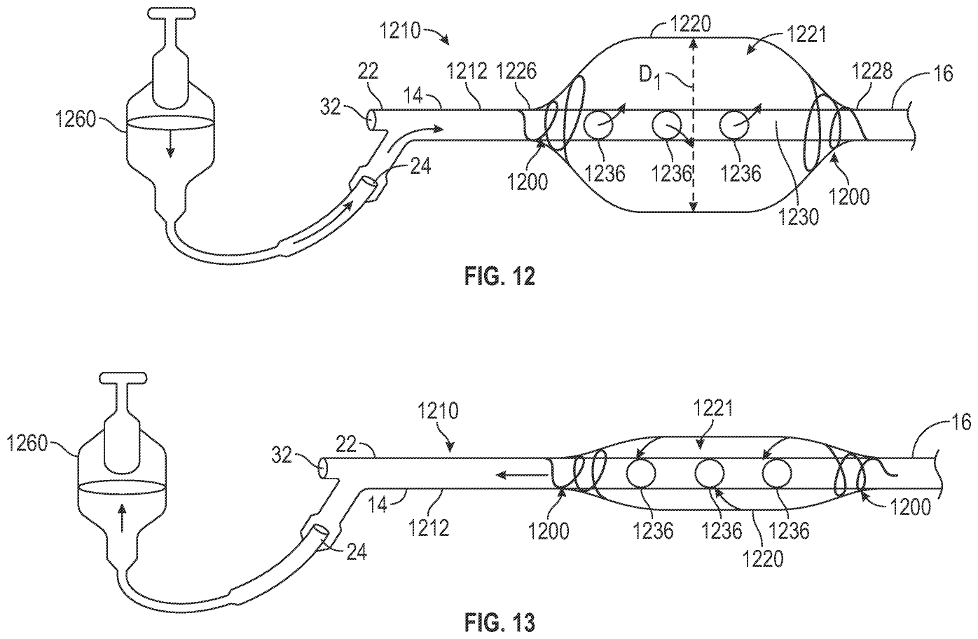

Referring now to the drawings, FIG. 1A illustrates an exemplary balloon catheter 10, according to an embodiment of the invention. As shown in FIG. 1A, the balloon catheter 10 includes an elongate shaft 12 and a balloon 20 disposed on a distal end 16 of the elongate shaft 12. The elongate shaft 12 may be made from a flexible catheter tubing, such as polyester, polyethylene, Nylon, or the like. The elongate shaft 12 is preferably tubular and extends between a proximal end 14 and the distal end 16. The proximal end 14 may attach to a hub 22. The hub 22 includes an inflation port 24, which then connects to a source of inflation fluid, such as an indeflator 1260 (shown in FIGS. 12 and 13), a syringe, or the like.

As shown in the drawings, the balloon 20 includes a proximal end 26 and a distal end 28. The proximal end 26 defines a proximal leg (e.g., a proximal neck portion of the balloon 20), and the distal end 28 defines a distal leg (e.g., a distal neck portion or a tip of the balloon 20). The proximal end 26 and the distal end 28 of the balloon 20 are each affixed to the elongate shaft 12. For example, the proximal end 26 and the distal end 28 of the balloon 20 may be bonded to the elongate shaft 12 such that a seal is created, and the inflation fluid is unable to escape out of the balloon 20. The balloon 20 defines a balloon chamber 21 in which the inflation fluid may enter to inflate the balloon 20 and may exit to deflate the balloon 20, as detailed further below. When the balloon 20 is fully inflated (as shown in FIG. 1A), a fully inflated profile shape of the balloon 20 includes a generally cylindrical working portion with an inflated diameter located between a pair of conical end portions. The conical end portions extend from the proximal end 26 and from the distal end 28, respectively. When the balloon 20 is deflated, the balloon 20 includes a deflated profile shape. The deflated profile shape of the balloon 20 may have several pleats that allow the balloon 20 to be wrapped around the elongate shaft 12 to reduce a profile of the balloon 20 to facilitate advancement of the balloon catheter 10 into a patient. When the balloon 20 is inflated, the balloon 20 may extend or expand in a circumferential direction and in a longitudinal direction.

In the embodiment of FIG. 1A, the elongate shaft 12 includes a dual lumen shaft. For example, the elongate shaft 12 includes an inflation lumen 30 for the passage of inflation fluid from the inflation port 24, and a wire guide lumen 32 to accommodate a wire guide 34. The inflation lumen 30 and the wire guide lumen 32 are coaxial. In this way, the wire guide lumen 32 is disposed within the inflation lumen 30. The inflation lumen 30 terminates distally from the distal end 28 of the balloon 20. The inflation lumen 30 includes one or more apertures 36. In the exemplary embodiment, the inflation lumen 30 includes three apertures 36. In some examples, the inflation lumen 30 may include three or more apertures 36. The use of three or more apertures 36 helps to drain the inflation fluid from the balloon chamber 21 faster as compared to balloon catheters with less than three apertures. The inflation lumen 30 can have three or more apertures 36 without sacrificing the structural integrity of the inflation lumen 30 or the balloon 20 by the use of one or more biasing members (e.g., a spring system) that maintains the structural integrity of the inflation lumen 30 as more apertures are added compared to balloon catheters without the benefit of the present disclosure. The one or more apertures 36 can have an aperture diameter in a range of ten thousandths of an inch to forty-five thousandths of an inch (0.0010 inches to 0.0045 inches). The one or more biasing members detailed below enable one or more apertures 36 to have a larger diameter as compared to balloons without the benefit of the present disclosure by maintaining the structural integrity of the inflation lumen 30 even while there are larger apertures 36 and less structure of the inflation lumen 30.

The inflation fluid is supplied through the inflation lumen 30 and through the one or more apertures 36 into the balloon chamber 21 to inflate the balloon 20. In some examples, the distal end 16 of the elongate shaft 12 may end distally of the proximal end 26 of the balloon 20 such that the inflation lumen 30 terminates distally from the proximal end 26 of the balloon 20 and proximal the proximal conical end portion of the balloon 20. In this way, the wire guide lumen 32 may extend through the balloon 20 and terminate distally from the distal end 28 of the balloon 20. In such examples, the inflation fluid exits the distal end of the inflation lumen 30 into the balloon chamber 21.

The wire guide 34 extends through the wire guide lumen 32 and may exit the balloon catheter 10 to aid in cannulation or perform some other function. The inflation fluid (e.g., water and/or saline) for inflation of the balloon 20 is supplied via the elongate shaft 12 through the inflation lumen 30 and into the balloon chamber 21, as detailed further below with reference to FIGS. 12 and 13.

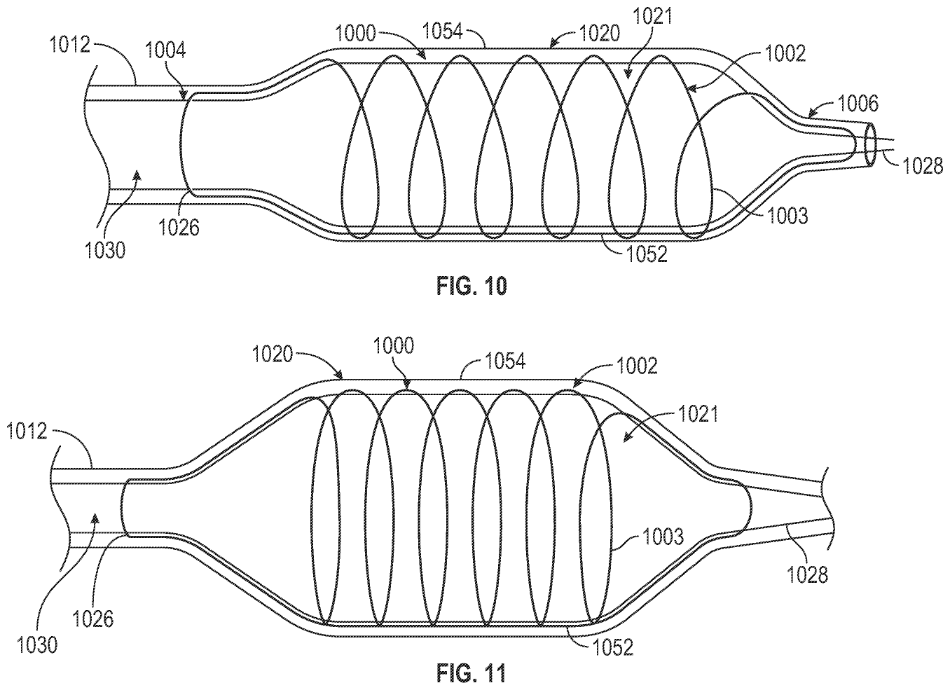

In some examples, the balloon 20 includes an inner balloon layer and an outer balloon layer, as discussed further below with reference to FIGS. 10 and 11. The balloon 20 may, of course, include any number of layers, as desired. Further, the balloon 20 may include any shape and/or size, as desired, for performing a balloon angioplasty.

FIG. 1B is a schematic view of the balloon 20 of FIG. 1A in a deflated state, according to an aspect of the disclosure. After the balloon 20 is inflated and expands, the elasticity of the balloon 20 prevents the balloon 20 from returning to the original shape of the balloon 20 when the balloon 20 is deflated. FIG. 1B shows that the balloon 20 forms wings 38 when the balloon 20 is deflated. The wings 38 are flat, lateral portions of the deflated balloon that project laterally outward beyond the catheter. In this way, the balloon 20 becomes wrinkled or otherwise bunched and the wings that form due to the bunching may damage the artery wall as the deflated balloon is advanced through the arterial system into the desired position for inflation, as detailed above.

FIG. 1C is a schematic side view of another balloon 120 in a deflated state, according to another embodiment. FIG. 1C shows the balloon 120 in a deflated state. The balloon 120 includes many of the same or similar components as the balloon 20 and may be used in the balloon catheter 10 of FIG. 1A. The balloon 120 is coupled to an elongate shaft 112 and extends between a proximal end 126 and a distal end 128. When the balloon 120 is deflated, the balloon 120 forms wings 138 and the balloon 120 twists or otherwise wraps around the elongate shaft 112. As detailed above, when the balloon 120 twists around the elongate shaft 112, the elongate shaft 112 weakens and a stiffness of the elongate shaft 112 is reduced.

FIG. 1D is a schematic view of the balloon 120 of FIG. 1C in a vessel 140 during a balloon angioplasty. The vessel 140 may be an artery or a vein of a patient. FIG. 1D shows the balloon 120 in a deflated state. The elongate shaft 112 extends through a hub or access sheath 122 and a wire guide 134 extends therethrough. FIG. 1D shows the elongate shaft 112 being retrieved or retracted from the vessel 140 through the access sheath 122. In this way, a portion of the balloon 120 has been retracted and is inside the access sheath 122 and any wings that were formed in that portion of the balloon 120 have been flattened by the access sheath 122. A winged portion 123 of the balloon 120 remains in the vessel 140 outside of the access sheath 122. The winged portion 123 includes the wings 138. FIG. 1D shows that the wings 138 become bunched such that the winged portion 123 of the balloon 120 is unable to be retracted into the access sheath 122. Thus, the balloon 120 becomes impeded and is unable to be fully retracted through the access sheath 122.

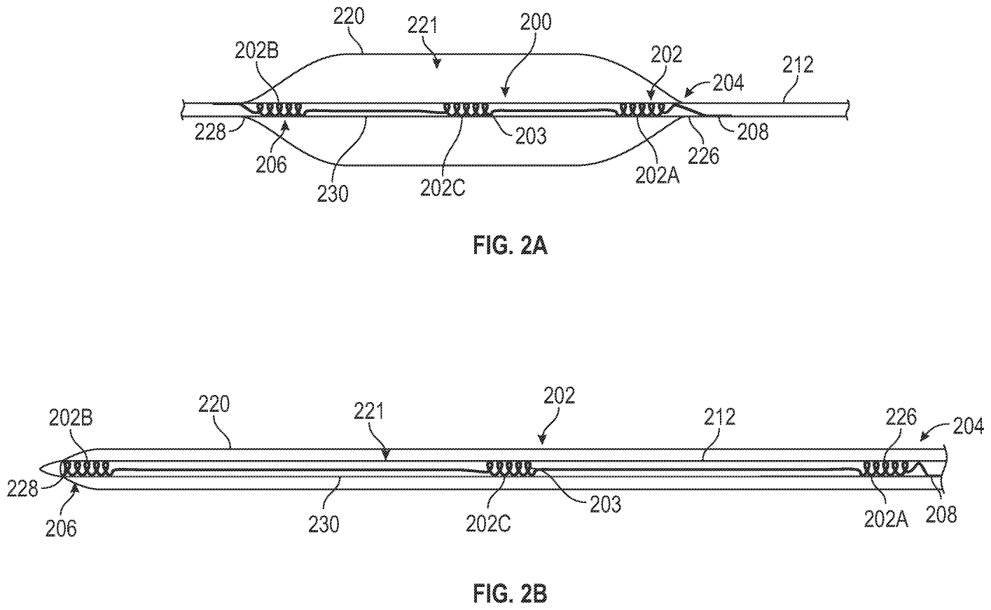

FIG. 2A is a schematic view of a balloon 220, according to another embodiment. The balloon 220 may be used in the balloon catheter 10 of FIG. 1A. As shown in FIG. 2, the balloon 220 is coupled to an elongate shaft 212 and extends between a proximal end 226 and a distal end 228. The balloon 220 includes a biasing member 200. As used herein, a “biasing member” includes a resilient, rigid, semi-rigid, flexible, or elastic member, and may be formed of any material, such as, for example, metals, polymers, plastics, elastomers, composite materials, rubber, or the like. The resilient nature of the biasing member enables the biasing member to move from a biased state to a non-biased state. The biased state is an original state or form of the biasing member. The biasing member can move to the non-biased state in which the biasing member generates and stores mechanical energy. When the stored mechanical energy is released, the biasing member is biased or otherwise returns to the biased state. For example, when the biasing member is a tension spring (e.g., the spring is tightly coiled in the biased state), the biased state is when the biasing member is compressed, and the non-biased state is when the biasing member is extended or otherwise expands. In this way, tension causes the biasing member to extend from the biased state to the non-biased state and the biasing member generates and stores mechanical energy. When the tension is released (e.g., the mechanical energy stored by the biasing member is released), the biasing member returns to the biased state. Likewise, when the biasing member is a compression spring (e.g., the spring is loosely coiled in the biased state), the biased state is when the biasing member is extended, and the non-biased state is when the biasing member is compressed. In this way, compression causes the biasing member to compress from the biased state to the non-biased state and the biasing member generates and stores mechanical energy. When the compression is released (e.g., the mechanical energy stored by the biasing member is released), the biasing member returns to the biased state.

In the exemplary embodiments of the present disclosure, the biasing member 200 includes a spring. The biasing member 200 may, however, include any type of biasing member. When the biasing member 200 is a spring, the biasing member 200 can be made of a material having a tensile strength that is a low strength, a moderate strength, or a high strength based on an application of the biasing member 200 for different types of balloons. For example, for smaller or thinner balloons and elongate shafts, the biasing member 200 is made of a material having tensile strength that is a lower strength. For larger and thicker balloons and elongate shafts, the biasing member 200 is made of a material having tensile strength that is a higher strength. Less atmospheric pressure is needed to inflate smaller or thinner balloons as compared to larger or thicker balloons. In this way, the material or the tensile strength of the biasing member 200 is selected such that the amount of atmospheric pressure for a particular balloon moves the biasing member 200 to the non-biased state.

In FIG. 2A, the biasing member 200 is coiled or otherwise wrapped around an inflation lumen 230 of the elongate shaft 212. The biasing member 200 may include one or more helical sections 202, also referred to as coiled sections, wrapped around the elongate shaft 212 within a balloon chamber 221 of the balloon 220. The one or more helical sections 202 include a first helical section 202A, a second helical section 202B, and a third helical section 202C each disposed around the elongate shaft 212 in the balloon chamber 221. Each helical section 202 includes one or more cells 203. The biasing member 200 may, of course, include any number of helical sections 202. The helical sections 202 in FIG. 2A are connected such that the helical sections 202 together form a single biasing member 200. In some examples, each helical section 202 may be a different biasing member 200 such that the helical sections 202 are not connected to one another. The biasing member 200 includes a proximal end 204 and a distal end 206. The distal end 206 is connected to the tip of the balloon 220, such as, at the distal end 228 of the balloon 220. The proximal end 204 is connected to a platform anchor 208 disposed on, and connected to, the elongate shaft 212. The platform anchor 208 is an extension of the biasing member 200 and forms a part thereof. In some examples, the platform anchor 208 is a separate component to which the biasing member 200 is coupled. In some examples, the platform anchor 208 is formed of the same or similar material as the elongate shaft 212 and may be made of polyester, polyethylene, Nylon, or the like. The platform anchor 208 provides an attachment mechanism for attaching the biasing member 200 to the elongate shaft 212. In this way, the biasing member 200 is prevented from slipping or otherwise rotating circumferentially about the elongate shaft 212 when the balloon 220 is inflated and/or deflated. The platform anchor 208 also helps to retain the force exerted by the expanding biasing member 200.

In the embodiment of FIG. 2A, when the balloon 220 is in a deflated state, the one or more helical sections 202 are tightly wrapped around the elongate shaft 212 such that the biasing member 200 is in a biased state (e.g., the biasing member 200 is compressed). As the inflation fluid is supplied to the balloon chamber 221, the pressure from the inflation fluid will cause the balloon 220 to inflate. At the same time, the pressure from the inflation fluid will cause tension in the helical sections 202 of the biasing member 200 such that the helical sections 202 expand and the biasing member 200 is considered to be in a non-biased state (e.g., the biasing member 200 is expanded or extended). For example, as the balloon 220 extends in the longitudinal direction, the helical sections 202 of the biasing member 200 correspondingly extend in the longitudinal direction such that a distance between the cells 203 increase, as detailed further below. In the non-biased state, the biasing member 200 stores mechanical energy, also referred to as stored negative force. As the balloon 220 is deflated (e.g., the inflation fluid is removed), the biasing member 200 compresses towards the biased state and releases the mechanical energy. For example, the helical sections 202 compress back to the compressed state such that the distance between the cells 203 decreases. In this way, the distal end 206 of the biasing member 200 pulls the distal end 228 of the balloon 220 longitudinally such that the balloon 220 retracts towards the deflated state. The compression of the biasing member 200 on the balloon 220 and on the elongate shaft 212 forces the inflation fluid through the one or more apertures 36 (FIG. 1A) faster as compared to balloon catheters without the benefit of the present disclosure. Further, the biasing member 200 may help drain or otherwise remove all the inflation fluid from the balloon 220 such that the balloon 220 returns to substantially an original shape.

FIG. 2B is a schematic view of the balloon 220 of FIG. 2A in a deflated state, according to an aspect of the disclosure. FIG. 2B shows that the balloon 220 returns to its original shape when the balloon 220 is deflated due to the biasing member 200, as detailed above. As shown in FIG. 2B, the balloon 220 is smooth and does not include any wings. In this way, the biasing member 200 prevents wings from forming such that the balloon 220 does not flatten or otherwise twist around the elongate shaft 212. Thus, the balloon 220 can be easily removed from the vessel and re-inserted as needed without damaging the vessel.

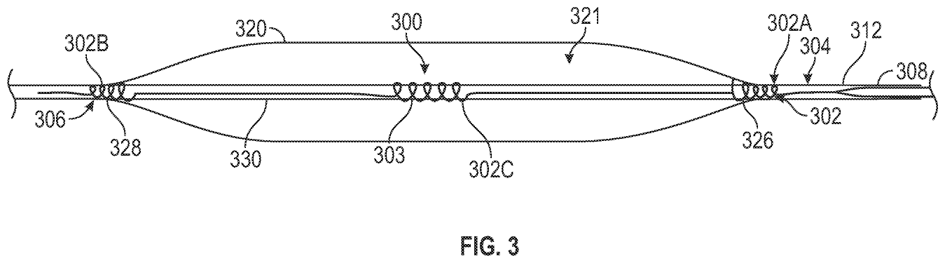

FIG. 3 is a schematic view of a balloon 320, according to another embodiment. The balloon 320 may be used in the balloon catheter 10 of FIG. 1A. As shown in FIG. 3, the balloon 320 is coupled to an elongate shaft 312 and extends between a proximal end 326 and a distal end 328. The balloon 320 includes a biasing member 300. The biasing member 300 is coiled or wrapped around the elongate shaft 312 (e.g., around an inflation lumen 330 of the elongate shaft 312). The biasing member 300 may include one or more helical sections 302 wrapped around the elongate shaft 312 within the balloon 320. For example, the biasing member 300 includes a first helical section 302A, a second helical section 302B, and a third helical section 302C each including one or more cells 303. The first helical section 302A is disposed around the elongate shaft 312 at the proximal end 326 of the balloon 320. The second helical section 302B is disposed around the elongate shaft 312 at the distal end 328 of the balloon 320. The third helical section 302C is disposed around the elongate shaft 312 within a balloon chamber 321 of the balloon 320. The biasing member 300 may, of course, include any number of helical sections 302, as detailed above. The biasing member 300 includes a proximal end 304 and a distal end 306. The distal end 306 is connected to the tip of the balloon 320, such as, at the distal end 328 of the balloon 320. The proximal end 304 is connected to a platform anchor 308 disposed on, and connected to, the elongate shaft 312.

When the balloon 320 is in a deflated state, the one or more helical sections 302 may be tightly wrapped around the elongate shaft 12 such that the biasing member 300 is in a biased state (e.g., the biasing member 300 is compressed). As the inflation fluid is supplied to the balloon chamber 321, the pressure from the inflation fluid will cause the balloon 320 to inflate. At the same time, the pressure from the inflation fluid will cause tension in the helical sections 302 of the biasing member 300 such that the helical sections 302 expand and the biasing member 300 is considered to be in a non-biased state (e.g., the biasing member 300 is expanded or extended). For example, as the balloon 320 extends in the longitudinal direction, the helical sections 302 of the biasing member 300 correspondingly extend in the longitudinal direction such that a distance between the cells 303 increase, as detailed further below. In the non-biased state, the biasing member 300 stores mechanical energy. As the balloon 320 is deflated (e.g., the inflation fluid is removed), the biasing member 300 compresses towards the biased state and releases the mechanical energy. For example, the helical sections 302 compress back to the compressed state such that the distance between the cells 303 decreases. In this way, the distal end 306 of the biasing member 300 pulls the distal end 328 of the balloon 320 longitudinally such that the balloon 320 retracts towards the deflated state. The compression of the biasing member 300 on the balloon 320 and on the elongate shaft 312 forces the inflation fluid through the one or more apertures 36 (FIG. 1A) faster as compared to balloon catheters without the benefit of the present disclosure. Further, the biasing member 300 may help drain all the inflation fluid from the balloon 320 such that the balloon 320 returns to substantially an original shape and wings are prevented from forming on the balloon 320.

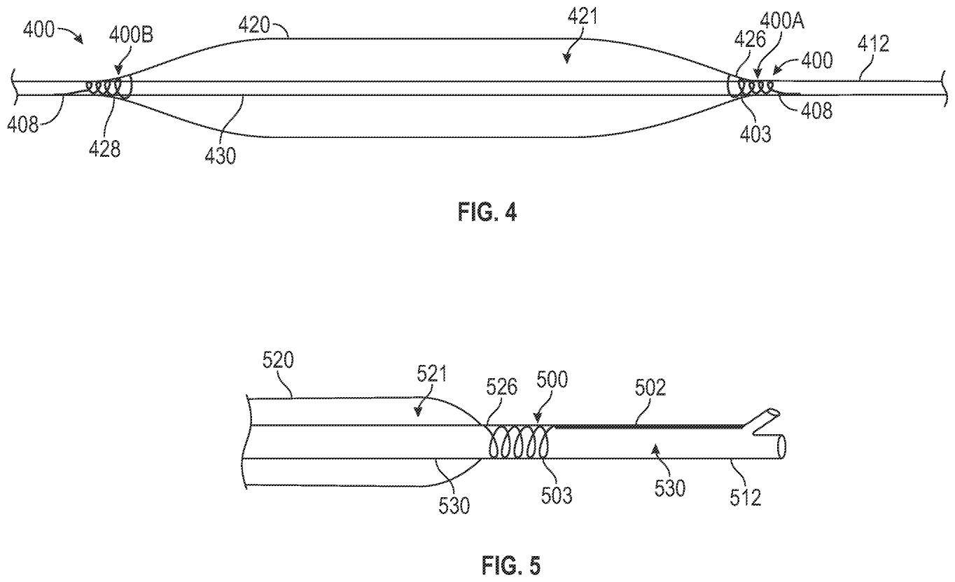

FIG. 4 is a schematic view of a balloon 420, according to another embodiment. The balloon 420 may be used in the balloon catheter 10 of FIG. 1A. As shown in FIG. 4, the balloon 420 is coupled to the elongate shaft 412 and extends between a proximal end 426 and a distal end 428. The balloon 420 includes a plurality of biasing members 400. The plurality of biasing members 400 includes a first biasing member 400A and a second biasing member 400B. The first biasing member 400A is disposed at the proximal end 426 of the balloon 420, and the second biasing member 400B is disposed at the distal end 428 of the balloon 420. Each biasing member 400A, 400B is coiled or wrapped around the elongate shaft 412 (e.g., around an inflation lumen 430 of the elongate shaft 412). The biasing members 400A, 400B may each define helical sections wrapped around the elongate shaft 412 within the balloon 420 and may include one or more cells 403. The second biasing member 400B is connected to a platform anchor 408 disposed on, and connected to, the elongate shaft 412 at the tip of the balloon 420, such as, at the distal end 428 of the balloon 420. The first biasing member 400A is connected to a platform anchor 408 disposed on, and connected to, the elongate shaft 412 at the proximal end 426 of the balloon 420.

When the balloon 420 is in a deflated state, the biasing members 400A, 400B may be tightly wrapped around the elongate shaft 412 such that the biasing members 400A, 400B are in a biased state (e.g., the biasing members 400A, 400B are compressed). As the inflation fluid is supplied to a balloon chamber 421 of the balloon 420, the pressure from the inflation fluid will cause the balloon 420 to inflate. At the same time, the pressure from the inflation fluid will cause tension in the biasing members 400A, 400B such that the biasing members 400A, 400B expand and the biasing members 400A, 400B are considered to be in a non-biased state (e.g., the biasing members 400A, 400B are expanded or extended). For example, as the balloon 420 extends in the longitudinal direction, the helical sections of the biasing members 400A, 400B correspondingly extend in the longitudinal direction such that a distance between the cells 403 increases. In the non-biased state, the biasing members 400A, 400B store mechanical energy. As the balloon 420 is deflated (e.g., the inflation fluid is removed), the biasing members 400A, 400B compress towards the compressed state and release the mechanical energy such that the distance between the cells 403 decreases. For example, the helical sections of the biasing members 400A, 400B compress back to the biased state. In this way, the second biasing member 400B pulls the distal end 428 of the balloon 420 and the first biasing member 400A pulls the proximal end 426 of the balloon 420 longitudinally such that the balloon 420 retracts towards the deflated state. The compression of the biasing members 400A, 400B on the balloon 420 and on the elongate shaft 412 forces the inflation fluid through the one or more apertures 36 (FIG. 1A) faster as compared to balloon catheters without the benefit of the present disclosure. Further, the biasing members 400A, 400B may help drain all the inflation fluid from the balloon 420 such that the balloon 420 returns to substantially an original shape and wings are prevented from forming on the balloon 420.

FIG. 5 is a schematic view of a balloon 520, according to another embodiment. The balloon 520 may be used in the balloon catheter 10 of FIG. 1A. As shown in FIG. 5, the balloon 520 is coupled to an elongate shaft 512 and extends between a proximal end 526 towards a distal end and includes a balloon chamber 521. The balloon 520 includes a biasing member 500. The biasing member 500 may be disposed around the elongate shaft 512 (e.g., around an inflation lumen 530 of the elongate shaft 512) at a location proximal to the proximal end 526 of the balloon 520. The biasing member 500 may be coupled to a platform anchor 502 disposed in the elongate shaft 512. The platform anchor 502 is coupled to an inner surface of the elongate shaft 512. The biasing member 500 may comprise any of the biasing members 200, 300, 400 of FIGS. 2A-4. In FIG. 5, the balloon 520 is in the deflated state, and thus the biasing member 500 is in the biased state (e.g., the biasing member 500 is compressed). As shown, the biasing member 500 includes tightly wrapped helical sections of cells 503. When the balloon 520 is in the deflated state, a longitudinal distance between the cells 503 is substantially zero.

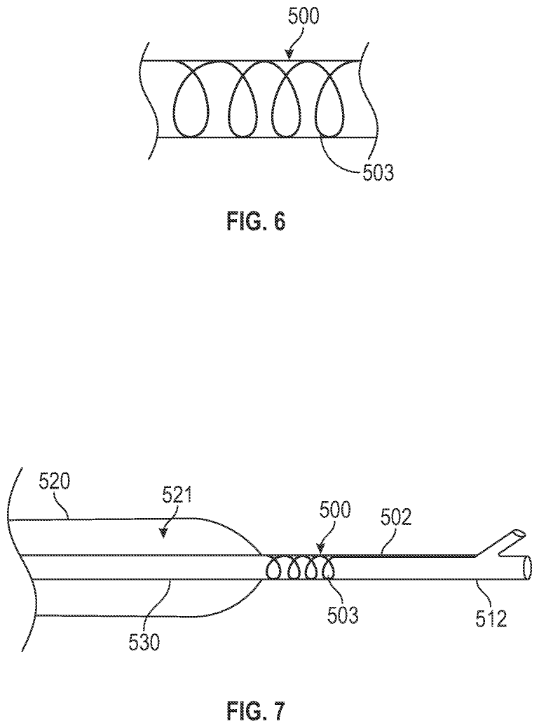

FIG. 6 is a schematic view of the biasing member 500 isolated from the balloon 520 in FIG. 5, according to an aspect of the disclosure. FIG. 7 is a schematic view of the balloon 520 in FIG. 5, according to an aspect of the disclosure. In FIGS. 6 and 7, the balloon 520 is in the inflated state, as detailed above. As shown in FIGS. 6 and 7, when the balloon 520 is inflated (e.g., inflation fluid is supplied to the balloon chamber 521 via the inflation lumen 530), the biasing member 500 expands in a longitudinal direction to the non-biased state. For example, the distance (x) between the cells 503 of the biasing member 500 increases (A increase) as compared to when the biasing member 500 is in the biased state. The biasing member 500 may also expand in a circumferential direction (as shown in FIG. 7). Thus, the biasing member 500 stores mechanical energy when the balloon 520 is inflated. According to one embodiment, the A increase in the distance (x) between the cells 503 of the biasing member 500 during balloon inflation indicates energy storage.

As the balloon 520 is deflated (e.g., the inflation fluid is removed), the biasing member 500 compresses towards the biased state and releases the mechanical energy. For example, as the biasing member 500 compresses, the biasing member 500 may pull the platform anchor 502 such that the balloon 520 and the elongate shaft 512 also compress. The compression of the biasing member 500 (e.g., and of the balloon 520 and the elongate shaft 512) forces the inflation fluid out of the balloon chamber 521 faster as compared to balloon catheters without the benefit of the present disclosure. Further, the biasing member 500 may help drain all the inflation fluid from the balloon 520 such that the balloon 520 returns to substantially an original shape. Thus, the biasing member 500 helps to reduce or prevent wings from forming in the balloon 520, as detailed above.

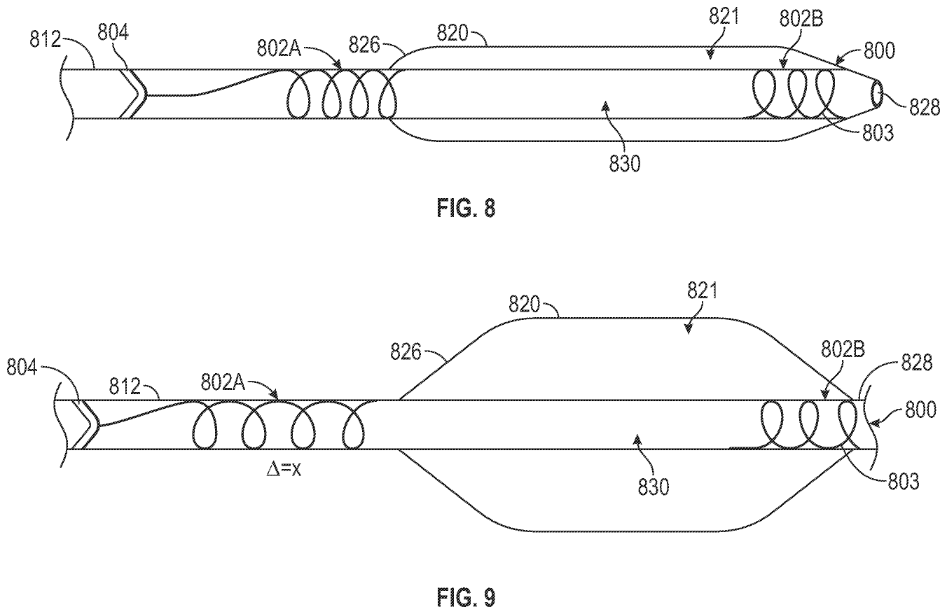

FIGS. 8 and 9 are schematic views of a balloon 820, according to another embodiment. The balloon 820 may be used in the balloon catheter 10 of FIG. 1A. As shown in FIG. 8, the balloon 820 is coupled to an elongate shaft 812 and extends between a proximal end 826 and a distal end 828. In FIG. 8, the balloon 820 is in a deflated state (i.e., Δ (or the distance x between adjacent cells 803 of a provided biasing member) equals zero), and in FIG. 9 the balloon 820 is in an inflated state (i.e., Δ equals to x (the distance between adjacent cells 803 of a provided biasing member)), as detailed above. As shown in FIGS. 8 and 9, the balloon 820 includes a biasing member 800. The biasing member 800 includes a first helical section 802A and a second helical section 802B each comprising one or more cells 803. The first helical section 802A and the second helical section 802B are wrapped around an inflation lumen 830 of the elongate shaft 812. The biasing member 800 is attached to a platform anchor 804. The platform anchor 804 is attached to the inner surface of the inflation lumen 830. The biasing member 800 may encompass any of the biasing members 200, 300, 400, 500 of FIGS. 2A-7. When the balloon 820 is in the deflated state (Δ=0) (FIG. 8), the biasing member 800 is in the biased state. When the balloon 820 is inflated to an inflated state (Δ=x) (FIG. 9), as detailed above, the inflation fluid forces the biasing member 800 to expand in a longitudinal direction such that a distance between the cells 803 increases. In this way, the biasing member 800 stores mechanical energy in the non-biased state.

When the balloon 820 is deflated (e.g., the inflation fluid is removed from a balloon chamber 821 of the balloon 820), as detailed above, the first helical section 802A and the second helical section 802B of the biasing member 800 each compress, as detailed above. The biasing member 800 may help drain all the inflation fluid from the balloon 820 such that the balloon 820 returns to substantially an original shape. Thus, the biasing member 800 helps to reduce or prevent wings from forming on the balloon 820, as detailed above.

FIGS. 10 and 11 are schematic views of a balloon 1020, according to another embodiment. The balloon 1020 may be used in the balloon catheter 10 of FIG. 1A. As shown in FIG. 10, the balloon 1020 is coupled to an elongate shaft 1012 and extends between a proximal end 1026 and a distal end 1028. In FIG. 10, the balloon 1020 is in a deflated state (Δ=0), and in FIG. 11 the balloon 1020 is in an inflated state (Δ=x), as detailed above. In the embodiment of FIGS. 10 and 11, the balloon 1020 includes a balloon commonly used for aortic applications, such as an aortic valve balloon. The balloon 1020 of FIGS. 10 and 11 includes an inner balloon layer 1052 and an outer balloon layer 1054. The inner balloon layer 1052 and the outer balloon layer 1054 each include an inflated diameter. The inflated diameter of the inner balloon layer 1052 is smaller than the inflated diameter of the outer balloon layer 1054.

As shown in FIGS. 10 and 11, the balloon 1020 includes a biasing member 1000. The biasing member 1000 includes a helical section 1002 having one or more cells 1003. The helical section 1002 is wrapped around the inner balloon layer 1052 of the balloon 1020. In this way, the biasing member 1000 is disposed between the inner balloon layer 1052 and the outer balloon layer 1054 of the balloon 1020. The biasing member 1000 includes a proximal end 1004 and a distal end 1006. The proximal end 1004 is wrapped around the proximal end 1026 of the inner balloon layer 1052 and the distal end 1006 is wrapped around the distal end 1028 of the inner balloon layer 1052. In this way, the biasing member 1000 forms a continuous member wrapped around the inner balloon layer 1052.

When the balloon 1020 is in the deflated state (Δ=0) (FIG. 10), the biasing member 1000 is in the biased state. When the balloon 1020 is inflated to an inflated state (Δ=x) (FIG. 11), as detailed above, the inflation fluid (e.g., supplied from an inflation lumen 1030) inflates the inner balloon layer 1052 and the outer balloon layer 1054. The inflation of the inner balloon layer 1052 causes the biasing member 1000 to expand in a circumferential direction. In this way, the biasing member 1000 stores mechanical energy in the non-biased state, as detailed above.

When the balloon 1020 is deflated (e.g., the inflation fluid is removed from a balloon chamber 1021 of the balloon 1020), as detailed above, the helical section 1002 of the biasing member 1000 compresses. The compression of the biasing member 1000 releases the mechanical energy and the biasing member 1000 pushes against or otherwise squeezes the inner balloon layer 1052. In this way, the inflation fluid is forced out of the balloon chamber 1021 faster as compared to balloon catheters without the benefit of the present disclosure. Further, the biasing member 1000 may help drain all the inflation fluid from the balloon 1020 and may pull the balloon 1020 longitudinally such that the balloon 1020 returns to substantially an original shape. Thus, the biasing member 1000 helps to reduce or prevent wings from forming on the balloon 1020.

FIGS. 12 and 13 illustrate an exemplary balloon catheter 1210, according to another embodiment. The balloon catheter 1210 includes many of the same or similar components as the balloon catheter 10 of FIG. 1A. As shown in FIGS. 12 and 13, the balloon catheter 1210 includes a balloon 1220 that is coupled to an elongate shaft 1212 and extends between a proximal end 1226 and a distal end 1228. FIG. 12 shows the balloon 1220 in a fully inflated state and FIG. 13 shows the balloon 1220 in a deflated state

An inflation lumen 1230 of the elongate shaft 1212 includes one or more apertures 1236. The one or more apertures 1236 are generally circular apertures. In FIGS. 12 and 13, the inflation lumen 1230 includes three or more apertures 1236. The inflation lumen 1230 can have a greater number of apertures compared to balloon catheters without the benefit of the present disclosure due to one or more biasing members 1200 that help to maintain the structural integrity of the inflation lumen 1230 and the balloon 1220 even when there is less structure due to the greater number of apertures. The balloon 1220 includes a balloon diameter D₁ that is defined as the diameter of the balloon 1220 at an axial center of the balloon 1220 when the balloon 1220 is in the fully inflated state.

FIGS. 12 and 13 show the source of inflation fluid is an indeflator 1260. The indeflator 1260 includes an inflation and deflation device used to inflate and deflate the balloon 1220. The indeflator 1260 is coupled to the inflation port 24. The indeflator 1260 stores inflation fluid (e.g., water and/or saline) and supplies the inflation fluid to a balloon chamber 1221 to inflate the balloon 1220. For example, when the indeflator 1260 is actuated in a first direction (as shown in FIG. 12), the indeflator 1260 generates positive pressure of the inflation fluid to supply the inflation fluid through the inflation lumen 1230 and into the balloon chamber 1221 to inflate the balloon 1220 (as indicated by the arrows in FIG. 12). The inflation fluid is supplied through the one or more apertures 1236 into the balloon chamber 1221. The indeflator 1260 maintains the positive pressure such that the balloon 1220 remains inflated.

When the indeflator 1260 is actuated in a second direction (as shown in FIG. 13), the indeflator 1260 generates negative pressure such that the inflation fluid is directed away from the balloon chamber 1221 towards the indeflator 1260, and the balloon 1220 deflates (as indicated by the arrows in FIG. 13). The inflation fluid exits the balloon chamber 1221 through the one or more apertures 1236 and flows through the inflation lumen 1230. In this way, the inflation fluid is removed from the balloon 1220 and the balloon 1220 is deflated.

As shown in FIGS. 12 and 13, the balloon 1220 includes one or more biasing members 1200. The one or more biasing members 1200 may encompass any of the biasing members of the embodiments as detailed above. In this way, when the balloon 1220 is inflated, the positive pressure of the inflation fluid from the indeflator 1260 overcomes the compression force of the biasing member 1200 in the biased state and the inflation fluid causes the biasing members 1200 to expand to the non-biased state when the balloon 1220 is inflated, as detailed above. For example, the force of the inflation fluid is greater than the force of the biasing members 1200 in the biased state. Thus, mechanical energy is stored in the biasing members 1200 (e.g., negative energy) and the positive force from the inflation fluid helps to retain the mechanical energy in the biasing members 1200 throughout the entire inflation time of the balloon 1220. When the balloon 1220 is deflated, the negative pressure causes the inflation fluid to exit the balloon 1220. In this way, the force of the biasing members 1200 is greater than the force of the inflation fluid, and the biasing members 1200 compress to the non-biased state. The compression of the biasing members 1200 releases the mechanical energy. The negative force of the biasing members 1200 is released and acts upon the inflation fluid in combination with the negative pressure. Thus, the inflation fluid exits the balloon chamber 1221 and the balloon 1220 deflates faster (e.g., accelerates the deflation of the balloon 1220) as compared to balloon catheters without the benefit of the present disclosure. The biasing members 1200 also help to ensure that the balloon 1220 is deflated to its original, deflated state. In this way, the biasing members 1200 helps to reduce or prevent the formation of wings on the balloon 1220. In some examples, the biasing members 1200 are expanded in the biased state and compressed in the non-biased state, as detailed above. Thus, the inflation fluid causes the biasing members 1200 to move from the biased state to the non-biased state such that the biasing members 1200 generate and store mechanical energy.

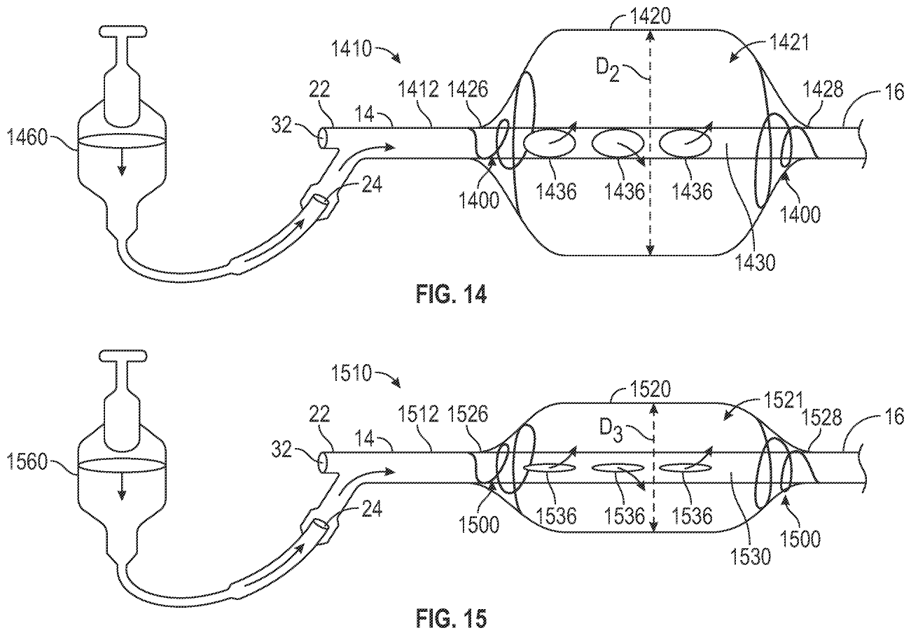

FIG. 14 illustrates an exemplary balloon catheter 1410, according to another embodiment. The balloon catheter 1410 in FIG. 14 is in a fully inflated state. The balloon catheter 1410 is substantially similar to the balloon catheter 1210 of FIG. 12. The balloon catheter 1410 includes one or more biasing members 1400, a balloon 1420 that is coupled to an elongate shaft 1412 and extends between a proximal end 1426 and a distal end 1428, and an inflation lumen 1430 having one or more apertures 1436. An indeflator 1460 is used to inflate and deflate the balloon 1420 with fluid into and out of a balloon chamber 1421.

The one or more apertures 1436 include three or more apertures 1436. The one or more apertures 1436 have a generally oval shape such that the one or more apertures 1436 include a major axis and a minor axis, the major axis being greater in length than the minor axis. The balloon 1420 has a balloon length that extends from the proximal end 1426 to the distal end 1428. The balloon length is in a range of twelve centimeters to twenty-four centimeters (12 cm to 24 cm). In this way, the balloon 1420 has a balloon length that is greater than balloons without the benefit of the present disclosure. The balloon 1420 has a balloon diameter D₂ that is defined as the diameter of the balloon 1420 at an axial center of the balloon 1420 when the balloon 1420 is in the fully inflated state. The balloon diameter D₂ is greater than the balloon diameter D₁ of the balloon 1220 of FIG. 12. The balloon diameter D₂ is greater than four millimeters (4 mm) when the balloon 1420 is in the fully inflated state.

The greater balloon length and the one or more biasing members 1400 enables the one or more apertures 1436 to have a generally oval shape (e.g., larger area than circular apertures), by providing a greater length to accommodate the one or more apertures 1436 and by the one or more biasing members 1400 maintaining the structural integrity of the inflation lumen 1430. The one or more biasing members 1400 also enable the balloon 1420 to have a greater balloon diameter as compared to balloons without the benefit of the present disclosure by maintaining the structural integrity of the balloon 1420. For example, merely making the apertures larger or the inflation lumen longer weakens the structural integrity of the inflation lumen, and the one or more biasing members 1400 enable the larger apertures and longer inflation lumen by maintaining the structural integrity, and, thus, reducing the influx and the efflux of the fluid into and out of the balloon 1420.

FIG. 15 illustrates an exemplary balloon catheter 1510, according to another embodiment. The balloon catheter 1510 in FIG. 15 is in a fully inflated state. The balloon catheter 1510 is substantially similar to the balloon catheters 1210, 1410 of FIGS. 12 and 14, respectively. The balloon catheter 1510 includes one or more biasing members 1500, a balloon 1520 that is coupled to an elongate shaft 1512 and extends between a proximal end 1526 and a distal end 1528, and an inflation lumen 1530 having one or more apertures 1536. An indeflator 1560 is used to inflate and deflate the balloon 1520 with fluid into and out of a balloon chamber 1521.

The one or more apertures 1536 include three or more apertures 1536. The one or more apertures 1536 are generally opened slits that include long, narrow openings or cuts in the inflation lumen 1530. The one or more apertures 1536 are narrower than the one or more apertures 1236, 1436 of FIGS. 12 and 14, respectively. The balloon 1550 has a balloon length that extends from the proximal end 1526 to the distal end 1528. The balloon length is less than twelve centimeters (12 cm). In this way, the balloon 1520 has a balloon length that is less than the balloon 1420 of FIG. 14. The balloon 1520 has a balloon diameter D₃ that is defined as the diameter of the balloon 1520 at an axial center of the balloon 1520 when the balloon 1520 is in the fully inflated state. The balloon diameter D₃ is less than the balloon diameter D₁ of the balloon 1220 of FIG. 12 and less than the balloon diameter D₂ of the balloon 1420 of FIG. 14. The balloon diameter D₃ is in a range of one millimeter to four millimeters (1 mm to 4 mm) when the balloon 1520 is in the fully inflated state. The one or more biasing members 1500 enable the one or more apertures 1536 to be generally opened slits in the inflation lumen 1530 that have a greater opened area than slits in balloon catheters without the benefit of the present disclosure by maintaining the structural integrity of the inflation lumen 1530, thereby allowing greater sized slits. For example, merely making the slits of current balloon catheters be opened (e.g., greater opened area) would weaken the structural integrity of the inflation lumen, and the one or more biasing members 1500 helps to maintain the structural integrity, thereby allowing the one or more apertures 1536 to be generally opened slits rather than closed slits, and, thus allowing more fluid to influx and efflux into and out of the balloon 1520, as compared to balloon catheters without the benefit of the present disclosure.

Further aspects are provided by the subject matter of the following clauses.

A balloon catheter comprising an elongate shaft having a proximal end and a distal end, a balloon attached to the elongate shaft at the distal end of the elongate shaft, the balloon including a balloon chamber for receiving inflation fluid, and one or more biasing members connected to the balloon, the one or more biasing members move to a non-biased state when the balloon is inflated and move to a biased state when the balloon is deflated such that the one or more biasing members cause the balloon to return to an original shape of the balloon.

The balloon catheter of the preceding clause, wherein the one or more biasing members are wrapped around the elongate shaft.

The balloon catheter of any preceding clause, wherein the one or more biasing members include a distal end connected to a distal end of the balloon, and wherein the one or more biasing members pull the distal end of the balloon when the balloon is deflated.

The balloon catheter of any preceding clause, further comprising a platform anchor connected to the elongate shaft, wherein the one or more biasing members are connected to the platform anchor.

The balloon catheter of any preceding clause, wherein the one or more biasing members include one or more helical sections having one or more cells.

The balloon catheter of any preceding clause, wherein the one or more helical sections are disposed within the balloon chamber.

The balloon catheter of any preceding clause, wherein the one or more helical sections include a first helical section disposed at a proximal end of the balloon and a second helical section disposed at a distal end of the balloon.

The balloon catheter of any preceding clause, wherein the one or more helical sections include a third helical section disposed within the balloon chamber.

The balloon catheter of any preceding clause, wherein the balloon extends in a longitudinal direction when the balloon is inflated, and wherein a distance between the one or more cells increases or decreases when the balloon is inflated.

The balloon catheter of any preceding clause wherein the distance between the one or more cells decreases or increases when the balloon is deflated such that the one or more biasing members pull the balloon and the balloon retracts in the longitudinal direction.

The balloon catheter of any preceding clause, wherein the balloon includes an inner balloon layer and an outer balloon layer, and wherein the one or more biasing members are wrapped around the inner balloon layer.

The balloon catheter of any preceding clause, wherein the one or more biasing members expand in a circumferential direction when the balloon is inflated.

The balloon catheter of any preceding clause, wherein the one or more biasing members compress against the inner balloon layer when the balloon is deflated.

The balloon catheter of any preceding clause, wherein positive pressure of the inflation fluid forces the one or more biasing members to move to the non-biased state such that the one or more biasing members store negative energy when the balloon is inflated.

The balloon catheter of any preceding clause, wherein negative pressure drains the inflation fluid from the balloon chamber and the stored negative energy of the one or more biasing members is released such that the one or more biasing members force the inflation fluid out of the balloon chamber when the balloon is deflated.

The balloon catheter of any preceding clause, wherein the elongate shaft includes three or more apertures for supplying the inflation fluid to the balloon chamber.

The balloon catheter of any preceding clause, wherein the elongate shaft includes one or more apertures, and the one or more apertures have an aperture diameter in a range of 0.0010 inches to 0.0045 inches.

The balloon catheter of any preceding clause, wherein the elongate shaft includes one or more apertures, and the one or more apertures have a generally circular shape.

The balloon catheter of any preceding clause, wherein the elongate shaft includes one or more apertures, and the one or more apertures have a generally oval shape.

The balloon catheter of any preceding clause, wherein the balloon has a balloon length that extends from a proximal end of the balloon to a distal end of the balloon, and the balloon length is in a range of 12 centimeters to 24 centimeters.

The balloon catheter of any preceding clause, wherein the balloon has a balloon diameter defined at an axial center of the balloon, and the balloon diameter is greater than 4 millimeters.

The balloon catheter of any preceding clause, wherein the elongate shaft includes one or more apertures, and the one or more apertures are generally opened slits.

The balloon catheter of any preceding clause, wherein the balloon has a balloon length that extends from a proximal end of the balloon to a distal end of the balloon, and the balloon length is less than 12 centimeters.

The balloon catheter of any preceding clause, wherein the balloon has a balloon diameter defined at an axial center of the balloon when the balloon is in a fully inflated state, and the balloon diameter is in a range of 1 millimeters to 4 millimeters.

A balloon catheter comprising an elongate shaft having a proximal end and a distal end, a balloon attached to the elongate shaft at the distal end of the elongate shaft, the balloon including a balloon chamber for receiving inflation fluid, and one or more biasing members connected to the balloon and wrapped around the elongate shaft, the one or more biasing members include one or more helical sections having one or more cells, wherein the one or more biasing members move to a non-biased state when the balloon is inflated and move to a biased state when the balloon is deflated such that the one or more biasing members cause the balloon to return to an original shape of the balloon.

The balloon catheter of the preceding clause, wherein the one or more biasing members include a distal end connected to a distal end of the balloon, and wherein the one or more biasing members pull the distal end of the balloon when the balloon is deflated.

The balloon catheter of any preceding clause, further comprising a platform anchor connected to the elongate shaft, wherein the one or more biasing members are connected to the platform anchor.

The balloon catheter of any preceding clause, wherein the one or more helical sections are disposed within the balloon chamber.

The balloon catheter of any preceding clause, wherein the one or more helical sections include a first helical section disposed at a proximal end of the balloon and a second helical section disposed at a distal end of the balloon.

The balloon catheter of any preceding clause, wherein the one or more helical sections include a third helical section disposed within the balloon chamber.

The balloon catheter of any preceding clause, wherein the balloon extends in a longitudinal direction when the balloon is inflated, and wherein a distance between the one or more cells increases or decreases when the balloon is inflated.

The balloon catheter of any preceding clause, wherein the distance between the one or more cells decreases or increases when the balloon is deflated such that the one or more biasing members pull the balloon and the balloon retracts in the longitudinal direction.

The balloon catheter of any preceding clause, wherein positive pressure of the inflation fluid forces the one or more biasing members to move to the non-biased state such that the one or more biasing members store negative energy when the balloon is inflated.

The balloon catheter of any preceding clause, wherein negative pressure drains the inflation fluid from the balloon chamber and the stored negative energy of the one or more biasing members is released such that the one or more biasing members force the inflation fluid out of the balloon chamber when the balloon is deflated.

The balloon catheter of any preceding clause, wherein the elongate shaft includes three or more apertures for supplying the inflation fluid to the balloon chamber.

The balloon catheter of any preceding clause, wherein the elongate shaft includes one or more apertures, and the one or more apertures have an aperture diameter in a range of 0.0010 inches to 0.0045 inches.

The balloon catheter of any preceding clause, wherein the elongate shaft includes one or more apertures, and the one or more apertures have a generally circular shape.

The balloon catheter of any preceding clause, wherein the elongate shaft includes one or more apertures, and the one or more apertures have a generally oval shape.

The balloon catheter of any preceding clause, wherein the balloon has a balloon length that extends from a proximal end of the balloon to a distal end of the balloon, and the balloon length is in a range of 12 centimeters to 24 centimeters.

The balloon catheter of any preceding clause, wherein the balloon has a balloon diameter defined at an axial center of the balloon, and the balloon diameter is greater than 4 millimeters.

The balloon catheter of any preceding clause, wherein the elongate shaft includes one or more apertures, and the one or more apertures are generally opened slits.

The balloon catheter of any preceding clause, wherein the balloon has a balloon length that extends from a proximal end of the balloon to a distal end of the balloon, and the balloon length is less than 12 centimeters.

The balloon catheter of any preceding clause, wherein the balloon has a balloon diameter defined at an axial center of the balloon when the balloon is in a fully inflated state, and the balloon diameter is in a range of 1 millimeters to 4 millimeters.

A balloon catheter comprising an elongate shaft having a proximal end and a distal end, a balloon attached to the elongate shaft at the distal end of the elongate shaft, the balloon including a balloon chamber for receiving inflation fluid, and the balloon including an inner balloon layer and an outer balloon layer, and one or more biasing members connected to the balloon and wrapped around the inner balloon layer, the one or more biasing members include one or more helical sections having one or more cells, wherein the one or more biasing members expand when the balloon is inflated and compress when the balloon is deflated such that the one or more biasing members force the inflation fluid out of the balloon chamber.

The balloon catheter of the preceding clause, wherein the one or more helical sections are disposed between the inner balloon layer and the outer balloon layer.

The balloon catheter of any preceding clause, wherein the one or more biasing members expand in a circumferential direction when the balloon is inflated.

The balloon catheter of any preceding clause, wherein the one or more biasing members compress against the inner balloon layer when the balloon is deflated.

The balloon catheter of any preceding clause, wherein positive pressure of the inflation fluid forces the one or more biasing members to expand such that the one or more biasing members store negative energy when the balloon is inflated.

The balloon catheter of any preceding clause, wherein negative pressure drains the inflation fluid from the balloon chamber and the stored negative energy of the one or more biasing members is released such that the one or more biasing members force the inflation fluid out of the balloon chamber when the balloon is deflated.

The balloon catheter of any preceding clause, wherein the elongate shaft includes three or more apertures for supplying the inflation fluid to the balloon chamber.

The balloon catheter of any preceding clause, wherein the elongate shaft includes one or more apertures, and the one or more apertures have an aperture diameter in a range of 0.0010 inches to 0.0045 inches.

The balloon catheter of any preceding clause, wherein the elongate shaft includes one or more apertures, and the one or more apertures have a generally circular shape.

The balloon catheter of any preceding clause, wherein the elongate shaft includes one or more apertures, and the one or more apertures have a generally oval shape.

The balloon catheter of any preceding clause, wherein the balloon has a balloon length that extends from a proximal end of the balloon to a distal end of the balloon, and the balloon length is in a range of 12 centimeters to 24 centimeters.

The balloon catheter of any preceding clause, wherein the balloon has a balloon diameter defined at an axial center of the balloon, and the balloon diameter is greater than 4 millimeters.

The balloon catheter of any preceding clause, wherein the elongate shaft includes one or more apertures, and the one or more apertures are generally opened slits.

The balloon catheter of any preceding clause, wherein the balloon has a balloon length that extends from a proximal end of the balloon to a distal end of the balloon, and the balloon length is less than 12 centimeters.

The balloon catheter of any preceding clause, wherein the balloon has a balloon diameter defined at an axial center of the balloon when the balloon is in a fully inflated state, and the balloon diameter is in a range of 1 millimeters to 4 millimeters.

Only exemplary embodiments of the present invention and but a few examples of its versatility are shown and described in the present disclosure. It is to be understood that the present invention is capable of use in various other combinations and environments and is capable of changes or modifications within the scope of the inventive concept as expressed herein.

Although the foregoing description is directed to the preferred embodiments of the invention, other variations and modifications will be apparent to those skilled in the art, and may be made without departing from the spirit or scope of the invention. Moreover, features described in connection with one embodiment of the invention may be used in conjunction with other embodiments, even if not explicitly stated above.

1. A balloon catheter comprising:
an elongate shaft having a proximal end and a distal end;
a balloon attached to the elongate shaft at the distal end of the elongate shaft, the balloon including a balloon chamber for receiving inflation fluid; and
one or more biasing members connected to the balloon, the one or more biasing members move to a non-biased state when the balloon is inflated and move to a biased state when the balloon is deflated such that the one or more biasing members cause the balloon to return to an original shape of the balloon.

2. The balloon catheter of claim 1, wherein the one or more biasing members are wrapped around the elongate shaft.

3. The balloon catheter of claim 1, wherein the one or more biasing members include a distal end connected to a distal end of the balloon, and wherein the one or more biasing members pull the distal end of the balloon when the balloon is deflated.

4. The balloon catheter of claim 1, further comprising a platform anchor connected to the elongate shaft, wherein the one or more biasing members are connected to the platform anchor.

5. The balloon catheter of claim 1, wherein the one or more biasing members include one or more helical sections having one or more cells.

6. The balloon catheter of claim 5, wherein the one or more helical sections are disposed within the balloon chamber.

7. The balloon catheter of claim 5, wherein the one or more helical sections include a first helical section disposed at a proximal end of the balloon and a second helical section disposed at a distal end of the balloon.

8. The balloon catheter of claim 7, wherein the one or more helical sections include a third helical section disposed within the balloon chamber.

9. The balloon catheter of claim 5, wherein the balloon extends in a longitudinal direction when the balloon is inflated, and wherein a distance between the one or more cells increases or decreases when the balloon is inflated.

10. The balloon catheter of claim 9, wherein the distance between the one or more cells decreases or increases when the balloon is deflated such that the one or more biasing members pull the balloon and the balloon retracts in the longitudinal direction.

11. The balloon catheter of claim 1, wherein the balloon includes an inner balloon layer and an outer balloon layer, and wherein the one or more biasing members are wrapped around the inner balloon layer.

12. The balloon catheter of claim 11, wherein the one or more biasing members expand in a circumferential direction when the balloon is inflated.

13. The balloon catheter of claim 12, wherein the one or more biasing members compress against the inner balloon layer when the balloon is deflated.

14. The balloon catheter of claim 1, wherein positive pressure of the inflation fluid forces the one or more biasing members to move to the non-biased state such that the one or more biasing members store negative energy when the balloon is inflated.

15. The balloon catheter of claim 14, wherein negative pressure drains the inflation fluid from the balloon chamber and the stored negative energy of the one or more biasing members is released such that the one or more biasing members force the inflation fluid out of the balloon chamber when the balloon is deflated.

16. The balloon catheter of claim 1, wherein the elongate shaft includes three or more apertures for supplying the inflation fluid to the balloon chamber.

17. The balloon catheter of claim 1, wherein the elongate shaft includes one or more apertures, and the one or more apertures have an aperture diameter in a range of 0.0010 inches to 0.0045 inches.

18. The balloon catheter of claim 1, wherein the elongate shaft includes one or more apertures, and the one or more apertures have a generally circular shape.

19. The balloon catheter of claim 1, wherein the elongate shaft includes one or more apertures, and the one or more apertures have a generally oval shape.

20. The balloon catheter of claim 1, wherein the balloon has a balloon length that extends from a proximal end of the balloon to a distal end of the balloon, and the balloon length is in a range of 12 centimeters to 24 centimeters.

21. The balloon catheter of claim 20, wherein the balloon has a balloon diameter defined at an axial center of the balloon, and the balloon diameter is greater than 4 millimeters.

22. The balloon catheter of claim 1, wherein the elongate shaft includes one or more apertures, and the one or more apertures are generally opened slits.

23. The balloon catheter of claim 22, wherein the balloon has a balloon length that extends from a proximal end of the balloon to a distal end of the balloon, and the balloon length is less than 12 centimeters.

24. The balloon catheter of claim 1, wherein the balloon has a balloon diameter defined at an axial center of the balloon when the balloon is in a fully inflated state, and the balloon diameter is in a range of 1 millimeters to 4 millimeters.

25. A balloon catheter comprising:
an elongate shaft having a proximal end and a distal end;
a balloon attached to the elongate shaft at the distal end of the elongate shaft, the balloon including a balloon chamber for receiving inflation fluid; and
one or more biasing members connected to the balloon and wrapped around the elongate shaft, the one or more biasing members include one or more helical sections having one or more cells, wherein the one or more biasing members move to a non-biased state when the balloon is inflated and move to a biased state when the balloon is deflated such that the one or more biasing members cause the balloon to return to an original shape of the balloon.

26. The balloon catheter of claim 25, wherein the one or more biasing members include a distal end connected to a distal end of the balloon, and wherein the one or more biasing members pull the distal end of the balloon when the balloon is deflated.

27. The balloon catheter of claim 25, further comprising a platform anchor connected to the elongate shaft, wherein the one or more biasing members are connected to the platform anchor.

28. The balloon catheter of claim 25, wherein the one or more helical sections are disposed within the balloon chamber.

29. The balloon catheter of claim 25, wherein the one or more helical sections include a first helical section disposed at a proximal end of the balloon and a second helical section disposed at a distal end of the balloon.

30. The balloon catheter of claim 29, wherein the one or more helical sections include a third helical section disposed within the balloon chamber.

31. The balloon catheter of claim 25, wherein the balloon extends in a longitudinal direction when the balloon is inflated, and wherein a distance between the one or more cells increases or decreases when the balloon is inflated.

32. The balloon catheter of claim 31, wherein the distance between the one or more cells decreases or increases when the balloon is deflated such that the one or more biasing members pull the balloon and the balloon retracts in the longitudinal direction.

33. The balloon catheter of claim 25, wherein positive pressure of the inflation fluid forces the one or more biasing members to move to the non-biased state such that the one or more biasing members store negative energy when the balloon is inflated.

34. The balloon catheter of claim 33, wherein negative pressure drains the inflation fluid from the balloon chamber and the stored negative energy of the one or more biasing members is released such that the one or more biasing members force the inflation fluid out of the balloon chamber when the balloon is deflated.

35. The balloon catheter of claim 25, wherein the elongate shaft includes three or more apertures for supplying the inflation fluid to the balloon chamber.

36. The balloon catheter of claim 25, wherein the elongate shaft includes one or more apertures, and the one or more apertures have an aperture diameter in a range of 0.0010 inches to 0.0045 inches.

37. The balloon catheter of claim 25, wherein the elongate shaft includes one or more apertures, and the one or more apertures have a generally circular shape.

38. The balloon catheter of claim 25, wherein the elongate shaft includes one or more apertures, and the one or more apertures have a generally oval shape.

39. The balloon catheter of claim 25, wherein the balloon has a balloon length that extends from a proximal end of the balloon to a distal end of the balloon, and the balloon length is in a range of 12 centimeters to 24 centimeters.

40. The balloon catheter of claim 39, wherein the balloon has a balloon diameter defined at an axial center of the balloon, and the balloon diameter is greater than 4 millimeters.

41. The balloon catheter of claim 25, wherein the elongate shaft includes one or more apertures, and the one or more apertures are generally opened slits.

42. The balloon catheter of claim 41, wherein the balloon has a balloon length that extends from a proximal end of the balloon to a distal end of the balloon, and the balloon length is less than 12 centimeters.

43. The balloon catheter of claim 25, wherein the balloon has a balloon diameter defined at an axial center of the balloon when the balloon is in a fully inflated state, and the balloon diameter is in a range of 1 millimeters to 4 millimeters.

44. A balloon catheter comprising:
an elongate shaft having a proximal end and a distal end;
a balloon attached to the elongate shaft at the distal end of the elongate shaft, the balloon including a balloon chamber for receiving inflation fluid, and the balloon including an inner balloon layer and an outer balloon layer; and
one or more biasing members connected to the balloon and wrapped around the inner balloon layer, the one or more biasing members include one or more helical sections having one or more cells, wherein the one or more biasing members expand when the balloon is inflated and compress when the balloon is deflated such that the one or more biasing members force the inflation fluid out of the balloon chamber.

45. The balloon catheter of claim 44, wherein the one or more helical sections are disposed between the inner balloon layer and the outer balloon layer.

46. The balloon catheter of claim 44, wherein the one or more biasing members expand in a circumferential direction when the balloon is inflated.

47. The balloon catheter of claim 46, wherein the one or more biasing members compress against the inner balloon layer when the balloon is deflated.

48. The balloon catheter of claim 44, wherein positive pressure of the inflation fluid forces the one or more biasing members to expand such that the one or more biasing members store negative energy when the balloon is inflated.

49. The balloon catheter of claim 48, wherein negative pressure drains the inflation fluid from the balloon chamber and the stored negative energy of the one or more biasing members is released such that the one or more biasing members force the inflation fluid out of the balloon chamber when the balloon is deflated.

50. The balloon catheter of claim 44, wherein the elongate shaft includes three or more apertures for supplying the inflation fluid to the balloon chamber.

51. The balloon catheter of claim 44, wherein the elongate shaft includes one or more apertures, and the one or more apertures have an aperture diameter in a range of 0.0010 inches to 0.0045 inches.

52. The balloon catheter of claim 44, wherein the elongate shaft includes one or more apertures, and the one or more apertures have a generally circular shape.

53. The balloon catheter of claim 44, wherein the elongate shaft includes one or more apertures, and the one or more apertures have a generally oval shape.

54. The balloon catheter of claim 44, wherein the balloon has a balloon length that extends from a proximal end of the balloon to a distal end of the balloon, and the balloon length is in a range of 12 centimeters to 24 centimeters.

55. The balloon catheter of claim 54, wherein the balloon has a balloon diameter defined at an axial center of the balloon, and the balloon diameter is greater than 4 millimeters.

56. The balloon catheter of claim 44, wherein the elongate shaft includes one or more apertures, and the one or more apertures are generally opened slits.

57. The balloon catheter of claim 56, wherein the balloon has a balloon length that extends from a proximal end of the balloon to a distal end of the balloon, and the balloon length is less than 12 centimeters.

58. The balloon catheter of claim 44, wherein the balloon has a balloon diameter defined at an axial center of the balloon when the balloon is in a fully inflated state, and the balloon diameter is in a range of 1 millimeters to 4 millimeters.
